(19) United States
(12) Patent Application Publication
(10) Pub. No.: US 2013/0205449 A1
(43) Pub. Date: Aug. 8, 2013

(54) Methods and Compositions for Production of Triploid Sterile Plants

(71) Applicant: Yi Li, Mansfield, CT (US)

(72) Inventor: Yi Li, Mansfield, CT (US)

(73) Assignee: University of Connecticut, Farmington, CT (US)

(21) Appl. No.: 13/567,520

(22) Filed: Aug. 6, 2012

Related U.S. Application Data
(60) Provisional application No. 61/515,170, filed on Aug. 4, 2011.

(51) Int. Cl. A01H 4/00

(52) U.S. Cl. CPC A01H 4/008
USPC 800/298; 435/430.1; 435/289.1

(57) ABSTRACT

Triploid sterile plant compositions and methods for production of triploid sterile plants are provided. The methods include regenerating a plant from an endosperm tissue by culturing the endosperm tissue in a series of different culture media containing certain plant growth regulators. Also provided are plants produced by the disclosed methods, as well as systems for production of the triploid sterile plants.

### PRIOR RELATED APPLICATION

The present application claims the benefit of priority to U.S. Provisional Application No. 61/515,170 filed Aug. 4, 2011, which is hereby incorporated by reference in its entirety.

### STATEMENT OF GOVERNMENT SUPPORT

This invention was made with government support under USDA Award Number 2006-38914-03519, USDA Award Number 2008-38914-19167, and USDA Award Number 2009-38914-19786 all awarded by the USDA. The government has certain rights in the invention.

### FIELD OF INVENTION

The present application is in the field of developing novel ornamental plants for horticultural purposes. In particular, methods of developing sterile cultivars of invasive ornamental plants are provided.

### BACKGROUND

Euonymus alatus (Thunb.) Sieb. commonly known as ‘burning bush’, is an extremely popular landscape plant in the United States due to its brilliant showy red leaves in fall. It is a deciduous shrub belonging to the family of Celastraceae and was introduced into the United States from northeastern/central China in 1860 for horticultural purposes (Chen et al., 2006, Plant Cell Rep. 25:1043-51; Dirr, 1998, Manual of Woody Landscape Plants, Stipes Publishing) As a result of its huge economic value and popularity, E. alatus has been extensively sold by nurseries, contributing significantly to the $16 billion U.S. ornamental horticulture industry (U.S. Department of Agriculture, 2005, Floriculture and Nursery Crops Outlook).

There are various cultivars of E. alatus available in the United States (e.g., ‘KoshoMayune,’ ‘Odom,’ ‘Pipzam,’ ‘Compactus,’ ‘Rudy Haag,’ and ‘Timber Creek’), but ‘Compactus’ is the most widely used cultivar because of its compact form and rounded shape. In Connecticut, annual sales of E. alatus ‘Compactus’ reached more than $5 million (Heffernan, 2005, “15 Invasive Plants of Value to the Green Industry Not Yet Banned In Connecticut,” Conn. Green Industries Publishing, Botsford, Conn.). E. alatus can survive in a wide range of soils and environmental conditions.

Although E. alatus is extremely popular, it also raises some concerns because it is seriously invasive due to its prolific seed production and effective seed dispersal by birds. A mature E. alatus plant produces tens of thousands of viable seeds a year. Birds and rainwater readily disperse those seeds to long distances, leading to the establishment of E. alatus plants in a variety of habitats. Once the plant is established, it can form dense thickets that displace native vegetation, posing a threat to the plant biodiversity (USDA, NRCS, 2005, The PLANTS Database, version 3.5). E. alatus has been reported to be invasive in 21 states in the U.S. (Ding et al., 2006, Biol. Invasions, 8:1439-50). Many states intend to ban the sale, planting, and propagation of E. alatus because of its invasiveness. However, as a result of the economic importance of E. alatus to the ornamental and landscape industries and its popularity among consumers, development of sterile, non-invasive E. alatus cultivars is in high demand (Gagliardi and Brand, 2007, Hort. Tech. 17:39-45).

The invasiveness of E. alatus is related to its high seed production, but sterility will eliminate the problem (Li et al., 2004, Crit. Rev. Plant Sci., 23:381-89). Sterile plants can be produced through conventional methods or by gene transfer approaches (Li et al., 2004, Crit. Rev. Plant Sci., 23:381-89). Although there are several methods available to produce sterile plants through gene transfer approaches (Chen et al., 2006, Plant Cell Rep., 25:1043-51; Chen et al., 2008, Acta Hort., 769:21-29; Li et al., 2004; Zheng et al., 2007, Plant Cell Rep., 26:1195-1203), the use of transgenic sterile ornamental plants may be limited (Li and Duan, 2011, Transgenic Horticultural Crops: Challenges and Opportunities, pp. 289-99). This is partially because of concerns over transgenic plants although some gene confinement technologies have been developed (Hong et al., 2009, Trends Biotech., 28:3-8; Kausch et al., 2010, Biofuels, 1:163-76; Li and Duan, 2011; Luo et al., 2007, Plant Biotech., 5:263-74).

At present, using non-transgenic sterile ornamental plants created by conventional procedures is much more desirable. One such procedure is to produce triploids, because triploid plants are sterile as a result of uneven chromosome division during meiosis (Ramsey and Schemske, 1998, Ann. Rev. Ecol. Sys., 29:467-501). Traditionally, triploids are produced by hybridization between tetraploids and diploids. That approach is time-consuming and technically difficult, especially for some woody species. Endosperm in angiosperms is naturally triploid, and in vitro culture of endosperm explants has been used as a method for producing triploid plants, although regeneration from endosperm tissues is often technically challenging (Thomas and Chaturvedi, 2008, Plant Cell Tissue, 93:1-14).

Therefore, what is needed in the art are triploid sterile ornamental plants that are seedless and non-invasive. Also needed are methods, compositions, and systems for producing the triploid sterile plants that are efficient, safe, and cost effective.

### SUMMARY OF THE DISCLOSURE

There is a great need for triploid sterile plants that are seedless and non-invasive. Such compositions and methods are provided herein. In particular, methods are provided for regenerating a triploid plant. In some embodiments, the methods involve culturing endosperm tissue in a callus induction medium for a time period effective to induce callus formation; culturing the callus in a shoot induction medium for a time period effective to induce shoot formation; culturing the shoot in a root induction medium for a time period effective to induce formation of a rooted triploid plant; thereby regenerating the triploid plant. In certain embodiments, one or more of the callus induction medium, the shoot induction medium, or the root induction medium include an effective amount of one or more plant growth regulators such as benzyladenine (BA), α-napthaleneacetic acid (NAA), indole-3-butyric acid (IBA), 2,4-dichlorophenoxy-acetic acid (2,4-D), or a combination thereof. In some embodiments, the culturing in a root induction medium includes culturing for a first time period in the presence of a plant growth regulator followed by the culturing for a second time period in the absence of an added plant growth regulator. In certain embodiments, the root induction medium used in the second time period further includes activated charcoal. In preferred embodiments, the culturing in each step is for a time period sufficient and effective to achieve the desired result of the step.

In certain embodiments, the plant is of the family Celastraceae. In certain embodiments, the plant is of the genus Euonymus. In certain embodiments, the plant is Euonymus alatus. In some embodiments, the Euonymus alatus plant is of the ‘Compactus’ cultivar. Plants produced by the described methods are provided herein, as well as systems for producing such plants using the described methods.

Using embodiments of the disclosed methods, it was determined that approximately 50% of immature endosperm explants and 14% of mature endosperm explants formed compact, green calli after culture in dark for eight weeks and then under light for four weeks on Murashige and Skoog (MS) medium supplemented with 2.2 μM BA and 2.7 μM NAA. Approximately 5.6% of the immature endosperm-derived calli and 13.4% of mature endosperm-derived calli initiated shoots within eight weeks after they were cultured on MS medium with 4.4 μM BA and 0.5 μM IBA. Eighty five percent of shoots rooted after culture on woody plant medium (WPM) containing 4.9 μM IBA for two weeks and then on hormone-free WPM medium containing 2.0 g/L activated charcoal for four weeks. Eight independently regenerated triploid plants were identified. Triploid plant regeneration rates observed were 0.42% from immature endosperm explants and 0.34% from mature endosperm explants, respectively, based on the number of endosperm explants cultured as described below. Because triploid plants cannot produce viable seeds, and are sterile and non-invasive, the triploid plant lines reported herein are useful for replacing the currently used invasive counterparts.

### BRIEF DESCRIPTION OF THE DRAWINGS

The following figures are exemplary embodiments of the present disclosure and are not intended to be limiting.

FIGS. 1A-1I are photographs of plant material from the various stages of triploid plant regeneration using the disclosed methods. Panel A is a photograph showing an immature endosperm explant isolated from immature fruit. Panel B is a photograph showing a green callus developed from an immature endosperm explant that was cultured initially in dark and then under light on a MS medium with 2.22 μM BA and 2.69 μM NAA. Panel C is a photograph showing an immature endosperm-derived callus forming adventitious buds on a MS medium with 4.44 μM BA and 0.49 mM IBA. Panel D is a photograph showing a mature endosperm-derived callus forming buds on a MS medium with 4.44 μM BA and 0.49 μM IBA. Panel E is a photograph showing shoots developed from immature endosperm-derived calli. Panel F is a photograph showing a rooted triploid plant regenerated from an immature endosperm-derived callus. Panel G is a photograph showing endosperm regenerated plants entering dormancy. The plants had yellow or red color leaves, inactive shoot apical, and lateral buds. Panel H is a photograph showing a new flush of growth observed on the plants that were cold treated at 4° C. for 3 months and then cultured at room temperature for 50 days; and Panel I is a photograph showing an acclimatized triploid plant growing in a pot containing Promix soil in the greenhouse.

FIGS. 2A to 2I are graphs showing flow cytometry histograms of E. alatus ‘Compactus’ diploid plants and endoderm-derived plants. The y axis of each graph is the number of nuclei counted, and the x axis is the linear fluorescence intensity (DNA content per nucleus). Panel A is a histogram of linear fluorescence intensity of nuclei from a diploid (2C) plant having the peak value of 197. Panel B is a histogram of linear fluorescence intensity of nuclei from a diploid (2C) plant that was derived from an embryonic callus tissue with the peak value of 200. Panel C is a histogram of linear fluorescence intensity of nuclei from a plant derived from endosperm, with the peak value of 200, indicating it is a diploid plant. Panels D to F are histograms of linear fluorescence intensity of nuclei extracted from three endosperm-derived triploid (3C) plants, T1, T2, and T3, with peak values of 307, 310, and 310, respectively. Panels G to I are histograms of linear fluorescence intensity of mixtures of nuclei from a diploid plant and three triploid plants, respectively. Panel G is a histogram from a mixture of leaf tissues from a diploid plant and the triploid T1 plant with the peak values of 183 and 281. Panel H is a histogram from a mixture of leaf tissues from a diploid plant and the triploid T2 plant with peak values of 201 and 306; and Panel I is a histogram of a mixture of leaf tissues from a diploid plant and the triploid T3 plant with peak values of 200 and 302. These data demonstrate that T1, T2, and T3 are triploids. The flow cytometer is basically a fluorescence particle counter, which measures fluorescence intensities in nuclei. The fluorescence intensity is proportional to the amount of the nuclear DNA to which the fluorescent stain is bound. A peak value of the fluorescence intensity reflects a mean of DNA content per nucleus. Peak values for the nuclear DNA contents from diploid plants of Euonymus alatus ‘Compactus’ were given the arbitrary value of 2C, and therefore, if the peak value of nuclear DNA content from an endosperm-derived Euonymus alatus ‘Compactus’ plant is 1.5-fold of that of the diploid's nuclear DNA, then the plant is triploid.

### DESCRIPTION

The present compositions and methods may be understood more readily by reference to the following detailed description of the preferred embodiments and the Examples included herein. However, before the present compositions and methods are disclosed and described, it is to be understood that the disclosed compositions and methods are not limited to specific cell types, specific conditions, or specific methods, etc., as such may, of course, vary, and the numerous modifications and variations therein will be apparent to those skilled in the art. It is also to be understood that the terminology used herein is for the purpose of describing specific embodiments only and is not intended to be limiting. It is further to be understood that unless specifically defined herein, the terminology used herein is to be given its traditional meaning as known in the relative art.

The present disclosure fulfills the need in the art by providing methods for successful production of triploid plants using endosperm tissues as explants. Since the first endosperm culture and regeneration protocol was reported in the early 1950s, attempts have been made to regenerate plants from endosperm tissues of a large number of plant species, but successful regeneration of buds/shoots from endosperm explants has been reported in 32 species only (Miyashita et al., 2009, Plant Cell Tissue Organ Cult., 98:291-301; Sun et al., 2011, Plant Cell Tissue Organ Cult., 104:23-29; Thomas and Chaturvedi, 2008, Plant Cell Tissue Organ Cult., 93:1-14). This suggests that plant regeneration from endosperm is difficult for many plant species. In general, it has been reported that one or more plant growth regulators are required for in vitro plant regeneration from immature endosperm. In most of the reports, callus induction media contained an auxin, preferably 2,4-D (reviewed by Thomas and Chaturvedi, 2008). In the present study as discussed in detail below, it was determined that E. alatus ‘Compactus’ immature endosperm explants cultured on the MS medium supplemented with various concentrations of BA and 2,4-D had loose, yellowish, and friable calli. However, green, compact calli were formed on the MS medium supplemented with BA and NAA. These data also show that, although 50% of immature endosperm explants could form calli, most of the endosperm-derived calli did not respond to different treatments of cytokinins (BA, zeatin, and kinetin) and auxins (IBA and IAA) and therefore failed to initiate buds. A small number of calli cultured on MS medium with 4.44 or 13.32 μM BA and 0.49 μM IBA produced buds, and the triploid plant regeneration rate was low, 0.42% (six triploid shoots regenerated from 1,440 immature endosperm explants). By comparison, when mature endosperm explants were used as explants, both callus induction rate and triploid plant regeneration rate were much lower, 14% and 0.34%, respectively. Similar results on shoot bud initiation were reported in Lonicera caerulea var. emphyllocalyx in which shoot primordial formation was observed on the MS media substituted only with 2.22 μMBA plus 0.49 μM IBA (10.0%) and 4.44 μM BA plus 0.49 μM or 4.92 μM IBA (3.3% and 2.7%, respectively), (Miyashita et al., 2009, Plant Cell Tissue Organ Cult. 98:291-301). Slightly higher callus induction and shoot regeneration efficiencies were obtained if E. alatus ‘Compactus’ immature endosperm explants were used. These observations demonstrate that it is difficult to regenerate plants from triploid tissues of E. alatus. Plants derived from endosperm tissues of diploid plant species are not always all triploids (reviewed by Thomas and Chaturvedi, 2008). In the present application, it was observed that many endosperm-derived plants were diploid rather than triploid although most of the endosperm-derived calli were found to be triploid (data not shown). It was also observed that plant regeneration from diploid embryo derived callus tissues was more efficient than from the triploid endosperm tissues (See, e.g., Table 3). Diploid plants have also been regenerated from endosperm cultures in other plant species like Azadirachta indica (Chaturvedi et al., 2003) and Actinidia chinensis (Gui et al., 1993, Euphytica, 71:57-62). Incomplete fertilization, chromosome loss, or development from maternal tissues have been proposed as possible reasons for the recovery of diploid plants from endosperm tissues (Gui et al., 1993). In Lonicera caerulea endosperm cultures, although most of the regenerated plants were hexaploid, an aneuploid plant has also been regenerated (Miyashita et al., 2009).

The present application describes the successful production of triploid plants from both immature and mature endosperm explants of E. alatus. No differences in plant morphology or growth and developmental patterns were observed between the diploid plants and the endosperm derived triploid plants produced by the methods described herein. Non-invasive triploid E. alatus varieties are ideal candidates to replace the currently used, highly invasive counterparts, which should be beneficial to both the ornamental industry and the consumers.

### Methods for Production of Triploid Sterile Plants

Methods are provided for regenerating a triploid plant of Euonymus. In some embodiments, the methods involve obtaining Euonymus endosperm tissue from a suitable source; culturing Euonymus endosperm tissue in a callus induction medium for a time period effective to induce callus formation; culturing the callus in a shoot induction medium for a time period effective to induce shoot formation; and culturing the shoot in a root induction medium for a time period effective to induce formation of a rooted triploid plant of Euonymus; thereby regenerating the triploid plant of Euonymus. In certain embodiments, one or more of the callus induction medium, the shoot induction medium, or the root induction medium contains an effective amount of one or more plant growth regulators. In certain embodiments, the plant growth regulators are benzyladenine (BA), α-napthaleneacetic acid (NAA), indole-3-butyric acid (IBA), 2,4-dichlorophenoxy-acetic acid (2,4-D), or a combination thereof.

In some embodiments, the culturing in a root induction medium includes culturing for a first time period in the presence of a plant growth regulator followed by the culturing for a second time period in the absence of an added plant growth regulator. In certain embodiments, the root induction medium used in the second time period further includes activated charcoal. In particular embodiments, the callus induction medium contains BA and NAA, the shoot induction medium contains BA and IBA, and the root induction medium contains IBA. In certain embodiments, the callus induction medium includes BA and NAA, the shoot induction medium includes BA and IBA, and the root induction medium used in the first root induction time period includes IBA.

In preferred embodiments, the culturing in each step is for a time period sufficient and effective to achieve the desired result of the step. In some embodiments, the culturing in callus induction medium step is for about eight to fourteen weeks, about eight to thirteen weeks, about nine to about thirteen weeks, about ten to about thirteen weeks, about eleven to twelve weeks, or about twelve weeks. In some embodiments, the culturing in shoot induction medium step is for about six to about ten weeks, about six to about nine weeks, about seven to ten weeks, about seven to nine weeks, about seven to eight weeks, about eight to nine weeks, or about eight weeks. In some embodiments, the culturing in the first root induction medium is for about one to four weeks, about one to three weeks, about one to two weeks, about two to three weeks, or about two weeks. In some embodiments, the culturing in the second root induction medium step is for about two to six weeks, about two to five weeks, about three to six weeks, about three to five weeks, about three to four weeks, about four to five weeks, or about four weeks. In one embodiment, the culturing in callus induction medium is for about twelve weeks, the culturing in shoot induction medium is for about eight weeks, the culturing in the first root induction medium is for about two weeks, and the culturing in the second root induction medium is for about four weeks.

In certain embodiments, the plant is of the family Celastraceae. In some embodiments, the plant is of the genus Celastrus, Euonymus, or Maytenus. In certain embodiments, the plant is an invasive plant such as American Bittersweet or Oriental Bittersweet. In certain embodiments, the plant is of the genus Euonymus. Euonymus may be referred to as spindle or spindle tree, and includes 170-180 species of deciduous and evergreen shrubs and small trees. In certain embodiments, the plant is Euonymus alatus. E. alatus grows to about 2.5 m tall (8.2 feet), often wider than tall. The stems are notable for their four corky ridges or “wings.” The flowers are greenish, borne over a long period in the spring. The fruit is a red aril enclosed by a four-lobed pink, yellow or orange capsule. The common name “burning bush” comes from the bright red fall color. It is a popular ornamental plant in gardens and parks due to its bright pink or orange fruit and attractive fall color. This plant is an invasive species of woodlands in eastern North America, and its importation and sale is prohibited in certain states. In some embodiments, the Euonymus alatus plant is of the ‘Compactus’, ‘Nodine,’ ‘Pipzam,’ ‘Kosho Mayune,’ ‘Odom,’ ‘Select,’ ‘Timber Creek,’ ‘Tures,’ or ‘Rudy Haag.” In certain embodiments, the Euonymus alatus plant is of the ‘Compactus’ cultivar.

Although the present methods have been described with respect to producing triploid plants of Euonymus, the methods are applicable to producing sterile triploid plants of all Celastraceae, although some minor modifications to the described conditions may be needed. The disclosed methods also can be referred to as seedless fruit methods, and applications of these seedless fruit methods can be used beyond invasive plants. For example, seedless fruit technology can be applied to improve quality and yield of fruit crops.

In some embodiments, the methods further include culturing the rooted triploid plant at 4° C. In certain embodiments, the culturing of the rooted triploid plant at 4° C. is in the dark and for a time period effective to break bud dormancy. In some embodiments, the time period is about one to about five months, about one to about four months, about two to about five months, about two to about four months, about two to about three months, about three to about four months, or about three months.

Euonymus plants, plant parts, or cells thereof obtained by the disclosed methods are provided. In certain embodiments, the plant is of the species Euonymus alatus. In certain embodiments, the plant is of the cultivar ‘Compactus.’

Methods of producing a triploid plant of the family Celastraceae are provided, including obtaining Celastraceae endosperm tissue; culturing the endosperm tissue in a callus induction medium for a time period effective to induce callus formation; culturing the callus in a shoot induction medium for a time period effective to induce shoot formation; culturing the shoot in a root induction medium for a time period effective to induce formation of a rooted triploid plant of Celastraceae, thereby producing the triploid plant. Plants, plant parts, and cells thereof obtained by these methods are provided. In certain embodiments, one or more of the callus induction medium, the shoot induction medium, or the root induction medium contains an effective amount of one or more plant growth regulators. The plant growth regulators may be BA, NAA, 2,4-D, IBA, or a combination thereof. In some embodiments, the culturing in a root induction medium step includes culturing for a first time period in the presence of a plant growth regulator followed by culturing for a second time period in the absence of an added plant growth regulator. In certain embodiments, the root induction medium used in the second time period further includes activated charcoal. In some embodiments, the callus induction medium contains BA and NAA, the shoot induction medium contains BA and IBA, and the root induction medium used in the first root induction time period contains IBA. Certain embodiments of the methods further include the step of culturing the rooted triploid plant at 4° C. This step may be in the dark and for a time period effective to break bud dormancy.

In one specific embodiment, the methods of producing a triploid plant include the following conditions:

1). Callus Induction Media: Culture Period: 12 Weeks

Medium: MS salts (Murashige and Skoog, 1962) with 100 mg/L myo-inositol, 2.0 mg/L glycine, 1.0 mg/L thiamine-HCl, 0.5 mg/L nicotinic acid, 0.5 mg/L pyridoxine-HCl, 0.3 g/L casein hydrolysate, 0.5 g/L 2-(N-morpholino) ethanesulfonic acid (MES), 30.0 g/L sucrose, and 7.0 g/L agar. pH 5.8.
Plant growth regulators: BA: 2.22 μM+NAA 2.69 μM

2). Shoot Induction Media: Culture Period: 8 Weeks

MS salts (Murashige and Skoog, 1962) with 100 mg/L myo-inositol, 2.0 mg/L glycine, 1.0 mg/L thiamine-HCl, 0.5 mg/L nicotinic acid, 0.5 mg/L pyridoxine-HCl, 0.3 g/L casein hydrolysate, 0.5 g/L 2-(N-morpholino) ethanesulfonic acid (MES), 30.0 g/L sucrose, and 7.0 g/L agar. pH 5.8.
Plant growth regulators: BA: 4.44 μM+IBA 0.49 μM

3). Root Induction Media (RM):

RM1-Culture period: 2 weeks

Half strength Woody Plant medium salts+50 mg/L myo-inositol, 1.0 mg/L glycine, 0.5 mg/L thiamine-HCl, 0.25 mg/L nicotinic acid, 0.25 mg/L pyridoxine-HCl, 20.0 g/L sucrose, 0.4 g/L ammonium nitrate, 4 g/L agar, 1.4 g/L phytagel. pH 5.2.

Plant growth regulator: IBA 4.9 μM.

RMI2-Culture period: 4 weeks

Half strength Woody Plant medium salts+50 mg/L myo-inositol, 1.0 mg/L glycine, 0.5 mg/L thiamine-HCl, 0.25 mg/L nicotinic acid, 0.25 mg/L pyridoxine-HCl, 20.0 g/L sucrose, 0.4 g/L ammonium nitrate, 4 g/L agar, 1.4 g/L phytagel, 2.0 g/L activated charcoal. pH 5.2.

4). Cold Treatment for 3 Months:

Half strength Woody Plant medium salts+50 mg/L myo-inositol, 1.0 mg/L glycine, 0.5 mg/L thiamine-HCl, 0.25 mg/L nicotinic acid, 0.25 mg/L pyridoxine-HCl, 20.0 g/L sucrose, 0.4 g/L ammonium nitrate, 4 g/L agar, 1.4 g/L phytagel, 2.0 g/L activated charcoal, pH 5.2.

The disclosed methods also comprise a number of variations and modifications. For example, the recited ranges in time periods for culturing steps may vary by at least +/− about 20% to +/− about 50% of the stated times. Similarly, the concentrations of various components of the culture media, such as the plant regulators, may vary from at least about 20% of the stated concentration to about five times the stated concentration. In some embodiments, the callus induction media includes BA at a concentration of about 0.5 μM to about 20 μM, about 1 μM to about 19 μM, or about 2 μM to about 18 μM. In some embodiments, the callus induction medium includes NAA at a concentration of about 1 μM to about 14 μM, about 2 μM to about 12 μM, or about 2.5 μM to about 11 μM. In some embodiments, the shoot induction media includes BA at a concentration of about 2.5 μM to about 15 μM, about 3.5 μM to about 14 μM, or about 4.0 μM to about 13 μM. In some embodiments, the callus induction media includes IBA at a concentration of about 0.35 μM to about 0.65 μM, about 0.45 μM to about 0.55 μM, or about 0.5 μM. In some embodiments, the first root induction media includes IBA at a concentration of about 3 μM to about 7 μM, about 4 μM to about 6 μM, or about 5 μM. In some embodiments, the second root induction media includes activated charcoal at a concentration of about 1.0 g/L to about 3.0 g/L, about 1.5.0 g/L to about 2.5 g/L, or about 2.0 g/L.

A system for regenerating a triploid plant is provided, that includes: a module for obtaining endosperm tissue from a suitable source; a module for culturing the endosperm tissue in a callus induction medium for a time period effective to induce callus formation; a module for culturing the callus in a shoot induction medium for a time period effective to induce shoot formation; and a module for culturing the shoot in a root induction medium for a time period effective to induce formation of a rooted triploid plant.

### EXAMPLES

Embodiments of the compositions, methods, and systems described herein are further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the invention. During the studies described the following examples, conventional procedures were followed, unless otherwise stated. Some of the procedures are described below for illustrative purpose.

### Example 1

### Plant Materials and Culture Conditions

### Plant Material

Immature fruits and mature fruits were collected in the first week of September and third week of November, respectively, from 10-year-old plants of E. alatus ‘Compactus’ grown in the Prides Corner Farms, Lebanon, Conn.

### Culture Media and Conditions.

Except for rooting, all the media described herein were composed of MS salts (Murashige and Skoog, 1962) with 100 mg/L myo-inositol, 2.0 mg/L glycine, 1.0 mg/L thiamine-HCl, 0.5 mg/L nicotinic acid, 0.5 mg/L pyridoxine-HCl, 0.3 g/L casein hydrolysate, 0.5 g/L 2-(Nmorpholino) ethanesulfonic acid, 30.0 g/L sucrose, and 7.0 g/L agar. The pH of all culture media was adjusted to 5.8 with KOH or HCl before addition of agar. All media were autoclaved at 121° C. for 20 minutes, and all the cultures, except for those specifically mentioned, were maintained at 23±2° C. under 35 to 45 μmol/m²s light provided by white cool fluorescent tube lamps over a 16-hour photoperiod.

### Example 2

Callus and Shoot Induction from Immature Endosperm Explants

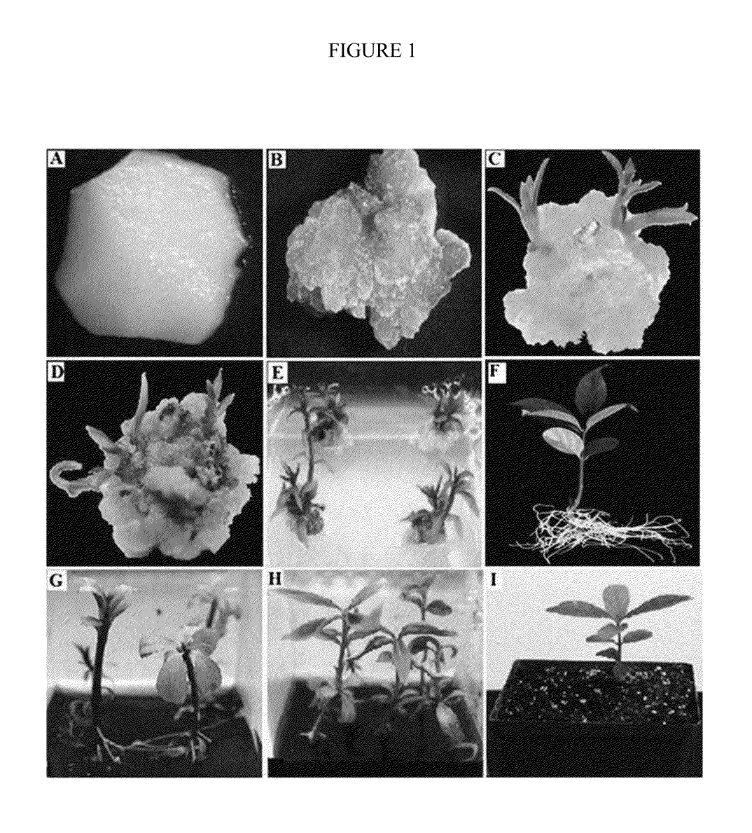

Endosperm tissues with seedcoats and embryos completely removed were used for triploid callus induction (FIG. 1A). Immature fruits of E. alatus ‘Compactus’ were thoroughly washed with running tap water for 30 minutes. The fruits were then surface-sterilized by immersion in 0.6% solution of sodium hypochlorite containing 0.1% Tween 20 (Sigma, St. Louis, Mo.) for 30 minutes and rinsed three times with sterile distilled water. The fruits were then soaked in 75% (v/v) ethanol and swirled for two minutes. After washing three times in sterile distilled water, immature seeds were isolated from the fruits.

Seed coats and embryos were removed, and the immature endosperm explants were cultured in disposable petri dishes (90×20 mm) containing MS medium supplemented with different combinations of four concentrations of BA (2.22, 4.44, 8.88, and 17.76 μM), three concentrations of NAA (2.69, 5.38, and 10.76 μM), and two concentrations of 2,4-dichlorophenoxy-acetic acid (2,4-D) (2.26 and 9.04 μM) to induce callus formation. Immature endosperm explants were also cultured on hormone-free MS medium as control. Thirty endosperm explants were cultured for each replicate at ten explants per petri dish, and each treatment had three replicates. Explants were cultured initially in the dark for eight weeks and then under light (15 to 20 μmol/m²s) for another four weeks. The cultures were transferred to fresh media every two weeks. Callus formation data were recorded at Week 12 of culture, and mean number of immature endosperm explants forming calli was calculated based on three replicates for each treatment. Callus induction rate was calculated by dividing the number of explants forming calli by the number of immature endosperm explants cultured. Mean callus induction rate was based on three replicates for each treatment.

After 12 weeks of culture, compact, greenish calli formed on six callus induction media supplemented with 2.22, 4.44, or 8.88 μM BA and 2.69 or 5.38 μM NAA were cut into pieces (approximately 0.5 cm×0.5 cm in size) and transferred to the petri dishes containing shoot induction media supplemented with 4.44 or 13.32 μM BA, 9.13 or 18.26 μM zeatin, and 9.30 or 18.60 μM kinetin plus 0.49 μM IBA or 0.57 μM indole-3-acetic acid (IAA). Callus segments also were cultured on hormone free MS medium as a control. Fifty callus tissues were cultured for each replicate with ten segments per petri dish. Each treatment had three replicates. All the cultures were transferred onto fresh media every two weeks, and the bud induction lasted for eight weeks. Bud (shoot bud) formation was recorded at Week 8 of culture. Bud induction rates were calculated by dividing the number of calli forming shoot buds by the number of calli cultured.

After the best media for callus and shoot induction were identified, a large number of immature endosperm explants were isolated from E. alatus ‘Compactus’ fruits and cultured on the MS medium containing 2.22 μM BA and 2.69 mM NAA for callus formation. Compact, green calli produced were then transferred onto the MS medium containing 4.44 μM BA and 0.49 μM IBA for shoot induction. Meanwhile, 100 immature embryos also were isolated and cultured on MS medium substituted with 2.22 μM BA and 2.69 μM NAA to produce calli. At Week 12 of culture, embryos forming calli were transferred to the MS medium containing 4.44 μM BA and 0.49 μM IBA for shoot induction. Callus induction rates were calculated based on the number of embryo/endosperm explants forming calli (observed at Week 12) divided by the number of embryo/endosperm explants cultured. Data on shoot formation were collected at Week 8 of culture. Shoot induction rates were calculated by dividing the number of calli that developed shoot buds by the number of calli cultured.

Plant growth regulators were essential for the immature endosperm of E. alatus to form callus. Callus was not induced on the explants cultured on hormone-free MS medium within 12 weeks of culture. Callus formation was observed on the surfaces of some explants after two to three weeks of culture on the medium containing BA and NAA or six weeks of culture on the medium containing BA and 2,4-D. All calli were white or cream in color and loose when cultured in dark, but those on NAA-containing media gradually turned green and compact after being transferred to light (FIG. 1B). Within 12 weeks of culture, significant differences in callus induction were observed among some media (Table 1). 2,4-D was less effective in callus induction than NAA when used in combination with the same concentration of BA. In addition, calli induced on 2,4-D-supplemented media were yellowish and loose even when they were maintained under light. The highest callus induction rate (51%) was observed on the medium supplemented with 2.22 mMBA plus 2.69 mM NAA).

TABLE 1

| | | | Number of | | |
|---|---|---|---|---|---|
| Concn of growth | | | explants | Mean callus | |
| regulator (μM) | | | forming calli | induction rate | Callus |
| BA | NAA | 2,4-D | (mean ± SE)^{y} | (% ± SE)^{x} | characteristics |
| 0 | 0 | 0 | 0 ± 0 | 0 ± 0 j^{w} | NA |
| 2.22 | 2.69 | — | 15.3 ± 1.2 | 51.1 ± 4.0 a | Compact and |
| | | | | | greenish |
| 4.44 | 2.69 | — | 12.0 ± 1.0 | 40.0 ± 3.3 b | Compact and |
| | | | | | greenish |
| 8.88 | 2.69 | — | 9.7 ± 0.7 | 32.2 ± 2.2 c | Compact and |
| | | | | | greenish |
| 17.76 | 2.69 | — | 3.3 ± 0.9 | 11.1 ± 2.9 fghi | Compact and |
| | | | | | greenish |
| 2.22 | 5.38 | — | 9.0 ± 1.1 | 30.0 ± 3.8 c | Compact and |
| | | | | | greenish |
| 4.44 | 5.38 | — | 6.3 ± 0.7 | 21.1 ± 2.2 de | Compact and |
| | | | | | greenish |
| 8.88 | 5.38 | — | 5.3 ± 0.3 | 17.8 ± 1.1 def | Compact and |
| | | | | | greenish |
| 17.76 | 5.38 | — | 4.3 ± 0.3 | 14.4 ± 1.1 efgh | Compact and |
| | | | | | greenish |
| 2.22 | 10.76 | — | 6.7 ± 0.3 | 22.2 ± 1.1 d | Compact and |
| | | | | | greenish |
| 4.44 | 10.76 | — | 5.7 ± 0.7 | 18.9 ± 2.2 de | Compact and |
| | | | | | greenish |
| 8.88 | 10.76 | — | 5.3 ± 0.7 | 17.8 ± 2.2 def | Compact and |
| | | | | | greenish |
| 17.76 | 10.76 | — | 2.7 ± 0.3 | 8.9 ± 1.1 ghi | Compact and |
| | | | | | greenish |
| 4.44 | — | 2.26 | 5.7 ± 0.3 | 18.9 ± 1.1 de | Loose and |
| | | | | | yellowish |
| 4.44 | — | 9.04 | 4.7 ± 0.7 | 15.5 ± 2.2 defg | Loose and |
| | | | | | yellowish |
| 17.76 | — | 2.26 | 2.3 ± 0.3 | 7.8 ± 1.1 hi | Loose and |
| | | | | | yellowish |
| 17.76 | — | 9.04 | 1.3 ± 0.3 | 4.4 ± 1.1 i | Loose and |
| | | | | | yellowish |

^{z}Each treatment had three replicates with 30 immature endosperm explants per replicate.
^{y}Callus formation was recorded at Week 12 of culture. Mean number of immature endosperm explants forming calli was derived from three replicates for each treatment.
^{x}Callus induction rate was calculated by dividing the number of explants forming calli by the number of immature endosperm explants cultered.
^{w}Data within a column not followed by the same letter are significantly different at P less than or equal to 0.05.

To induce bud formation, compact, green calli were cut into pieces and placed on a MS medium supplemented with different cytokinins (BA, zeatin, or kinetin) and auxins (IBA or IAA) at various concentrations. All calli grew well after subculture, enlarging in size and remaining green in color, but most of them did not produce buds for up to eight weeks after induction. Bud formation was observed only on the MS medium containing 4.44 or 13.32 μM BA plus 0.49 μM IBA T2 (Table 2). A small number of calli produced adventitious buds after three to four weeks of subculture (FIG. 1C). There was no significant difference in bud induction rates between the two BA treatments (Table 2).

Buds further developed into 1- to 2-cm long shoots during subsequent culture in Magenta boxes (FIG. 1E). However, the number of callus explants forming buds did not increase with the culture time. Both zeatin (at 9.13 or 18.26 μM) and kinetin (at 9.30 or 18.60 μM) failed to induce bud formation in the presence of 0.49 μM IBA.

TABLE 2

| | | | | | | Mean shoot |
|---|---|---|---|---|---|---|
| | | | | | Number of | bud |
| | | | | | calli forming | induction |
| Concn of growth regulator (μM) | | | | | shoot buds | rate |
| BA | Zeatin | Kinetin | IAA | IBA | (mean ± SE)^{y} | (% ± SE)^{x} |
| 0 | 0 | 0 | 0 | 0 | 0 ± 0 | 0 ± 0 b^{w} |
| 4.44 | — | — | — | 0.49 | 2.7 ± 0.3 | 5.3 ± 0.7 a |
| 13.32 | — | — | — | 0.49 | 3.0 ± 1.0 | 6.0 ± 2.0 a |
| 4.44 | — | — | 0.57 | — | 0 ± 0 | 0 ± 0 b |
| 13.32 | — | — | 0.57 | — | 0 ± 0 | 0 ± 0 b |
| — | 9.13 | — | — | 0.49 | 0 ± 0 | 0 ± 0 b |
| — | 18.26 | — | — | 0.49 | 0 ± 0 | 0 ± 0 b |
| — | — | 9.30 | — | 0.49 | 0 ± 0 | 0 ± 0 b |
| — | — | 18.60 | — | 0.49 | 0 ± 0 | 0 ± 0 b |

_{^{Z}}Each treatment had three replicates with 50 calli per replicate.
_{^{y}}Bud formation was recorded at Week 8 of culture. Mean number of calli forming shoot buds was based on three replicates for each treatment.
_{^{x}}Shoot bud induction rate was calculated by dividing the number of calli forming shoot buds by the number of calli cultured.
_{^{w}}Data within a column not followed by the same letter are significantly different at P less than or equal to 0.05.

During a four year period, a large number of immature endosperm explants have been isolated and cultured on the MS medium containing 2.22 μM BA and 2.69 μM NAA. With 1440 endosperms cultured, 676 of 1440 explants formed green calli after 12 weeks (Table 3). After the calli were subcultured on the MS medium supplemented with 4.44 μM BA and 0.49 μM IBA, 38 of them formed buds within eight weeks, yielding a shoot induction rate of 5.6% based on the number of calli cultured (Table 3).

TABLE 3

| | | Number of | Number of | Number of | Number of | Triploid |
|---|---|---|---|---|---|---|
| | Number of | explants | calli | independently | confirmed | plant |
| Explant | explants | forming | forming | regenerated | triploid | induction |
| type | cultured | calli | shoot buds | plant lines | plants | rates |
| Immature | 1440 | 676 (46.9%) | 38 (5.6%) | 37 | 6 | 0.42% |
| endosperm | | | | | | |
| Mature | 585 | 82 (14.0%) | 11 (13.4%) | 7 | 2 | 0.34% |
| endosperm | | | | | | |
| Immature | 100 | 86 (86%) | 75 (87.2%) | 69 | 0 | 0.00% |
| embryo | | | | | | |

Callus induction data were recorded at Week 12 of culture. Callus induction rates were calculated by dividing the number of explants forming calli by the number of embryo/endosperm explants cultured. Shoot bud induction data were recorded at Week 8 of culture. Shoot bud induction rates were calculated by dividing the number of calli showing shoot bud formation by the number of calli cultured. Triploid plant induction rates were calculated by dividing the number of independent triploid plants regenerated by the number of endosperm/embryo explants cultured.

### Example 3

Callus and Shoot Induction from Mature Endosperm Explants

Mature fruits were harvested, and seeds were isolated from the fruits, washed with tap water thoroughly, and then surface disinfected by submerging them in a solution of 1.2% sodium hypochlorite containing 0.1% Tween 20 for ten minutes followed by washing three times with sterile distilled water. Then, the seeds were further sterilized with a 0.6% solution of sodium hypochlorite containing 0.1% Tween 20 for 30 minutes followed by rinsing with sterile distilled water five times. Mature seeds were sterilized twice with sodium hypochlorite solution to minimize microbial contamination. After sodium hypochlorite sterilization and water rinsing, the seeds were soaked in 75% (v/v) ethanol for five minutes and washed five times with sterile water. Then they were transferred onto the MS medium (with 7.0 g/L agar), containing 100 μl/L Proclin 300 (Supelco, Bellefonte, Pa.), a preservative used in tissue culture media for the control of microorganism growth (Nagy et al., 2005).

It was observed that Proclin 300 worked well to minimize bacterial or fungal contaminations. The seeds were maintained on the MS medium at ten seeds per petri dish at 23° C. under light with a 16-hour photoperiod for one month and then in the dark at 4° C. for three months for two reasons. One is that individual contaminated seeds could be easily identified and removed during the first month culture. The other reason is that a prolonged incubation (additional three months) of seeds on MS medium made seedcoats soft and therefore could be removed when endosperm tissues were isolated.

At the end of four months, seedcoats and embryo tissues were carefully removed, and mature endosperm tissues were isolated and cultured on MS medium supplemented with 2.22 μM BA and 2.69 μM NAA to produce calli. Approximately 585 mature endosperm explants were cultured at 10 to 15 explants per petri dish. Culture conditions were same as those used for immature endosperm explants. At Week 12 of culture, observations were taken on callus induction, and compact greenish calli of approximately 0.75 cm×0.75 cm in size were transferred to the MS medium containing 4.44 μM BA and 0.49 μM IBA for shoot induction. Approximately ten calli were cultured per petri dish. Shoot formation results were recorded at Week 8 of culture on the shoot induction medium. All the cultures were transferred to fresh media every two to three weeks.

The results show that calli were also induced from mature endosperm explants of E. alatus ‘Compactus’ on MS medium containing 2.22 μM BA and 2.69 μM NAA, although the percentage was lower compared with that of the immature endosperm tissues (Table 3). After being transferred to bud induction medium (MS with 4.44 μM BA and 0.49 μM IBA), 11 of 82 mature endosperm-derived calli initiated buds within eight weeks (FIG. 1D; Table 3).

### Example 4

### Plant Regeneration and Breaking Dormancy

After shoot buds were formed from embryo or endosperm-derived calli, they were transferred together with their callus explants into Magenta boxes containing the MS medium supplemented with 4.44 μM BA and 0.49 μM IBA for further growth. When the shoots grew to 1 to 2 cm in height, they were excised from callus tissues and cultured on root induction Medium I, which included half strength WPM (Lloyd and McCown, 1980, Proc. Intl. Plant Prop. Soc., 30:421-427) with 2% sucrose, 0.04% ammonium nitrate, 0.4% agar, 0.14% phytagel (a blended agar and phytagel medium was used to produce a totally transparent medium so that initiation and growth of roots could be easily monitored), and 4.9 μM IBA (Clements, 2007, In vitro induction of polyploidy, rooting, and overcoming the dormancy limitations of Euonymus alatus ‘Compactus,” MS Thesis, Univ. of. Conn., p. 536)). Two weeks later, the shoots were transferred onto rooting Medium II (Clements, 2007), which was IBA-free rooting Medium I with 2 g/L activated charcoal (Kang et al., 2009). The pH of the rooting media was adjusted to 5.2 with KOH or HCl before the addition of gelling agents.

The root cultures were transferred to fresh medium every four weeks. It was observed that adventitious roots initiated from the base of the shoots four weeks after transfer, and 85% of the shoots developed into healthy plants with well-developed roots within 10 weeks of culture (FIG. 1F). After two months of culture on the rooting Medium II, the apical and lateral buds became dormant. Growth stopped and the leaves turned yellow to red in color (FIG. 1G). To break bud dormancy, the plants were transferred to a 4° C. incubator and maintained there for three months. The root cultures were transferred to fresh root induction Medium II every four weeks. After the cold treatment, plants were moved to a culture room to resume active growth, and two weeks later they started new growth. The newly growing plants generally reached 3 to 6 cm in height after two months (FIG. 1H).

### Example 5

### Production of Triploid Plants

Triploid lines from endosperm-derived plants were identified using flow cytometry techniques. Ploidy levels of individual plants derived from diploid embryo tissues were compared with those from endosperm tissues, which can be diploid, triploid, or tetraploid, using the Partec CyFlow Ploidy Analyser with DAPI as a fluorescent DNA stain. Ploidy determination was based on total DNA content per nucleus. Nuclei were extracted using the Partec Cystain PI absolute P kit according to the manufacturer's instructions. Approximately 0.5 cm×0.5 cm young leaves of the embryo- or endosperm-derived plants were chopped with a sharp razor blade in a 55-mm-diameter petri dish containing 500 mL nuclei extraction buffer. The extracts were filtered through a Partec 30 mm CellTrics disposable filter and stained with 2 mL staining buffer containing DAPI. The young leaves from embryo-derived plants were used as diploid controls (2C). Triploids (3C) were identified by comparing their linear fluorescence intensity (the DNA content per nucleus) with that of the diploid controls (2C). If an endosperm-derived plant had 50% more DNA per nucleus than that of embryo-derived plant (2C), the plant was considered a triploid.

Once triploid plants were identified, to reduce experimental variations or errors in ploidy level determination among different plant samples, diploid leaf tissues of E. alatus ‘Compactus’ were used as an internal standard. Leaf disks from a diploid plant and each triploid plant were mixed and chopped together to prepare nuclei. The nuclei mixture was further analyzed with flow cytometry. If the histogram had two distinct peaks with a ratio of 1:1.5 in the DNA content per nucleus, the triploid plant was verified.

The ploidy level of all triploid plants was further confirmed using a FACS Calibur Dual Laser Flow Cytometer (B.D. Immunocytometry Systems, San Jose, Calif.), and a different DNA stain, propidium iodide (PI), to quantify the DNA content per nucleus. PI has its excitation wavelength at 488 nm, and its emission wavelength at 562 to 588 nm, whereas the excitation and emission wavelengths of DAPI for the Partec CyFlow Ploidy Analyser are distinctly different. DAPI has its excitation at 372 nm and emission at 456 nm. Triploid plant lines were therefore further verified using two different DNA stains and two flow cytometers. Furthermore, to reduce experimental variations in ploidy level determination, leaf disks from a diploid plant also were mixed with leaf disks from each independent triploid line before nuclei were isolated. Again, if two distinct peaks with a ratio of 1:1.5 in the DNA content per nucleus were observed in the mixture, the triploidy was confirmed.

The ploidy level of each triploid plant derived from endosperm tissues was determined and verified at least three times based on the mentioned procedures with leaves harvested at different growth stages. Data described here were collected from a FACS Calibur Dual Laser Flow Cytometer with PI as the DNA stain. Triploid plant induction rates were derived by dividing the number of independent triploid plants regenerated by the number of endosperm explants cultured.

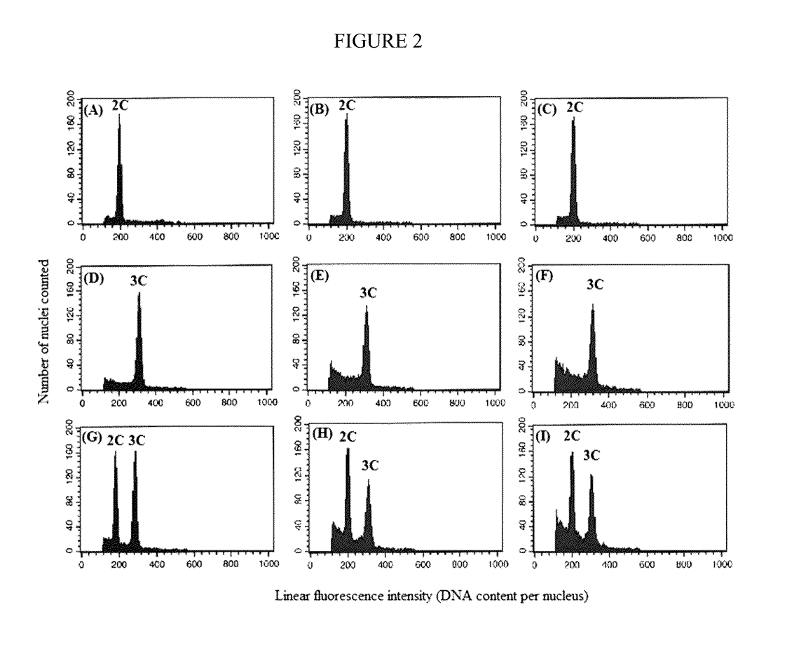

Based on 2,025 immature and mature endosperm explants cultured over two years, a total of 44 independently regenerated plant lines were obtained, 37 from immature endosperm explants and seven from the mature endosperm explants. Because of dormancy and cold treatment, eight of them did not grow well and eventually died. To identify triploids from the remaining 36 plant lines, ploidy levels were determined using the methods described previously. Twenty-eight plant lines had a DNA content per nucleus identical to that of F2 diploid controls (FIGS. 2A-C), and therefore, were diploids. However, eight independently regenerated triploid plants were identified, and the flow cytometry histograms for some of these triploid plants are shown in FIGS. 2D-F. When a diploid plant of E. alatus was used as an internal standard, a diploid DNA peak and a triploid DNA peak were observed in the same histogram. Peak values of DNA contents of the triploid nuclei were 1.5 times those of the diploid nuclei. Data from three representative triploid plants are shown in FIGS. 2G-I. The ploidy levels of all eight endosperm derived triploid plants were determined and verified three times using leaves harvested at different growth stages using two different ploidy analyzers and DNA staining dyes.

Haploid plants from tobacco pollen also were generated and compared to the ploidy levels of pollen-derived plants with diploid parental plants (data not shown). The results indicate that the methods used to determine the ploidy level of endosperm-derived plants were highly reliable. Therefore, the eight plants of E. alatus derived from endosperm tissues are triploids.

### Example 6

Triploid Plants were Capable of Normal Growth

Both triploid and diploid plants were acclimatized by gradual lid opening of Magenta boxes in three days before they were taken out of the boxes. Plants were gently washed under tap water to remove gelling agents, and they were then transplanted to 2-inch diameter pots with Promix potting soil (Premier Horticulture Inc.; PA). The potted plants were acclimatized for three to four weeks at room temperature and subsequently transferred to a greenhouse for further growth.

Triploid and diploid plants of E. alatus grew normally after transferring to the greenhouse, and more than 85% of the plants survived and produced three to four pairs of new leaves (FIG. 1I). The survived plants stopped growing four months after transfer to the greenhouse, and cold treatment was necessary to break bud dormancy and induce new growth. No visible differences were observed in growth rate and morphology between the triploid and diploid plants.

While the invention has been described with reference to preferred embodiments, it will be understood by those skilled in the art that various changes may be made and equivalents may be substituted for the elements thereof without departing from the scope of the invention. In addition, many modifications may be made to adapt the teaching of the invention to particular use, application, starting materials, production conditions, use conditions, composition, medium, size, and/or materials without departing from the essential scope thereof. Therefore, it is intended that the invention not be limited to the particular embodiments and best mode contemplated for carrying out this invention as described herein. The accompanying claims are intended to cover such modifications as would fall within the true scope and spirit of the present invention.

What is claimed is:

1. A method of regenerating a triploid plant of Euonymus, the method comprising:
culturing Euonymus endosperm tissue in a callus induction medium for a time period effective to induce callus formation;
culturing the callus in a shoot induction medium for a time period effective to induce shoot formation;
culturing the shoot in a root induction medium for a time period effective to induce formation of a rooted triploid plant of Euonymus;
thereby regenerating the triploid plant of Euonymus.

2. The method of claim 1, wherein one or more of the callus induction medium, the shoot induction medium, or the root induction medium comprises an effective amount of one or more plant growth regulators.

3. The method of claim 2, wherein the plant growth regulators are benzyladenine (BA), α-napthaleneacetic acid (NAA), indole-3-butyric acid (IBA), or a combination thereof.

4. The method of claim 1, wherein the culturing in a root induction medium comprises culturing for a first time period in the presence of a plant growth regulator followed by culturing for a second time period in the absence of a plant growth regulator.

5. The method of claim 4, wherein the root induction medium used in the second time period further comprises activated charcoal.

6. The method of claim 1, wherein the callus induction medium comprises BA and NAA, the shoot induction medium comprises BA and IBA, and the root induction medium comprises IBA.

7. The method of claim 4, wherein the callus induction medium comprises BA and NAA, the shoot induction medium comprises BA and IBA, and the root induction medium used in the first root induction time period comprises IBA.

8. The method of claim 4, wherein the culturing in callus induction medium is for about twelve weeks, wherein the culturing in shoot induction medium is for about eight weeks, wherein the culturing in the first root induction medium is for about two weeks, and wherein the culturing in the second root induction medium is for about four weeks.

9. The method of claim 6, wherein the callus induction medium comprises BA at a concentration of about 2 μm to about 18 μm, and comprises NAA at a concentration of about 2.5 μm to about 11 μm.

10. The method of claim 9, wherein the callus induction medium comprises BA at a concentration of about 2.2 μm and comprises NAA at a concentration of about 2.69 μm to about 11 μm.

11. The method of claim 6, wherein the shoot induction medium comprises BA at a concentration of about 4 μm to about 13 μm, and comprises IBA at a concentration of about 0.45 μm to about 0.55 μm.

12. The method of claim 11, wherein the shoot induction medium comprises BA at a concentration of about 4.44 μm, and comprises IBA at a concentration of about 0.49 μm.

13. The method of claim 6, wherein the root induction medium comprises IBA at a concentration of about 4.9 μm.

14. The method of claim 1, further comprising culturing the rooted triploid plant at 4° C.

15. The method of claim 14, wherein the culturing of the rooted triploid plant at 4° C. is in the dark and for a time period effective to break bud dormancy.

16. The method of claim 15, wherein the time period is about three months.

17. A Euonymus plant, plant part, or cell thereof obtained by the method of claim 1.

18. The Euonymus plant, plant part, or cell of claim 17, wherein the plant, plant part, or cell is of the species Euonymus alatus.

19. The Euonymus alatus plant, plant part, or cell of claim 18, wherein the plant, plant part, or cell is of the cultivar ‘Compactus.’

20. A system for regenerating a triploid Euonymus plant, comprising:
a module for culturing Euonymus endosperm tissue in a callus induction medium for a time period effective to induce callus formation;
a module for culturing the callus in a shoot induction medium for a time period effective to induce shoot formation; and
a module for culturing the shoot in a root induction medium for a time period effective to induce formation of a rooted triploid plant of Euonymus.

21. A method of regenerating a triploid plant of the family Celastraceae, the method comprising:
culturing Celastraceae endosperm tissue in a callus induction medium for a time period effective to induce callus formation;
culturing the callus in a shoot induction medium for a time period effective to induce shoot formation;
culturing the shoot in a root induction medium for a time period effective to induce formation of a rooted triploid plant of Celastraceae, thereby regenerating the triploid plant.

22. A Celastraceae plant, plant part, or cell thereof obtained by the method of claim 21.

23. The method of claim 21, wherein one or more of the callus induction medium, the shoot induction medium, or the root induction medium comprises an effective amount of one or more plant growth regulators, and wherein the plant growth regulators are benzyladenine (BA), α-napthaleneacetic acid (NAA), indole-3-butyric acid (IBA), or a combination thereof.

24. The method of claim 21, wherein the culturing in a root induction medium comprises culturing for a first time period in the presence of a plant growth regulator followed by the culturing for a second time period in the absence of an added plant growth regulator.

25. The method of claim 24, wherein the root induction medium used in the second time period further comprises activated charcoal.

26. The method of claim 21, further comprising culturing the rooted triploid plant at 4° C.
